(19) United States
(12) Patent Application Publication
(10) Pub. No.: US 2008/0275530 A1
(43) Pub. Date: Nov. 6, 2008

(54) Specialty Surgical Instrument

(75) Inventors: John K. Stevens, Stratham, NH (US); Paul Waterhouse, Selkirk (CA)

Correspondence Address: Marina Larson & Associates, LLC, re:VAI, P.O. Box 4928, Dillon, CO, 80435-4928, US

(73) Assignee: Visible Assets, Inc., Mississauga (CA)

(21) Appl. No.: 11/560,338

(22) Filed: Nov. 15, 2006

Related U.S. Application Data
(60) Provisional application No. 60/737,052, filed on Nov. 15, 2005.

(51) Int. Cl. A61N 1/00

(52) U.S. Cl. 607/60

(57) ABSTRACT

A metal surgical implant has a first antenna wrapped around an elongated portion, the first antenna electrically coupled with circuitry, all tuned to a frequency below 450 kilohertz. The assembly is substantially sterile. A second antenna may also be wrapped around the elongated portion, also electrically coupled with the circuitry. The implant may be a hip implant.

This application claims priority from U.S. application No. 60/737,052, filed Nov. 15, 2005, which application is incorporated herein by reference for all purposes.

### BACKGROUND

Implant surgery requires many, often hundreds, of specialized instruments. These instruments are most often produced with titanium or stainless steel with many varied sizes and shapes. They are expensive and are often accidentally lost or misplaced.

Much attention has been given in recent years to approaches intended to reduce how often such instruments are lost or misplaced. Likewise much attention has been given to trying to keep track of such instruments for accounting and billing purposes.

Not one of the approaches proposed heretofore has worked out well. In particular, the most commonly employed approach in recent years for keeping track of important or high-value items, namely a traditional RFID tag, is of no help at all with implant-type instruments, because they contain a lot of metal and the metal detunes the RFID tag or blocks its signal completely.

Given the fact that all RF-type approaches heretofore attempted have failed miserably due to detuning and signal blocking, it is counterintuitive to think that that any RF-type approach would be worth pursuing.

It would be very desirable to have an approach that would actually work with implant-type instruments, and that is consistent with the need for sterilization and that would have a suitable range (distance of function) for use in real medical environments.

### SUMMARY OF THE INVENTION

A metal surgical implant has a first antenna wrapped around an elongated portion, the first antenna electrically coupled with circuitry, all tuned to a frequency below 450 kilohertz. The assembly is substantially sterile. A second antenna may also be wrapped around the elongated portion, also electrically coupled with the circuitry. The implant may be a hip implant.

### DESCRIPTION OF THE DRAWINGS

The invention will be described with respect to a drawing in one FIGURE, of which:

FIG. 1 shows a plan view of an instrument assembly according to the invention.

### DETAILED DESCRIPTION

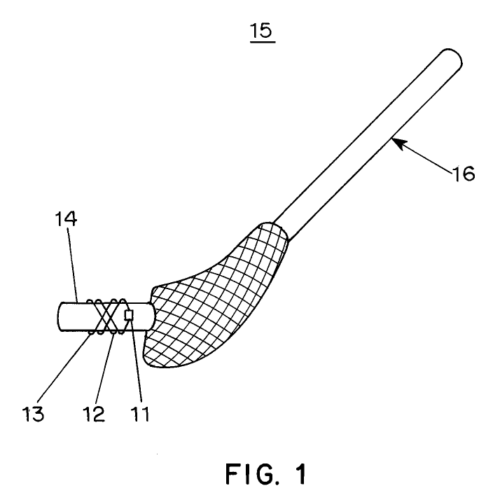

FIG. 1 shows an implant assembly 15 according to the invention. The instrument 16 has a rasp area shown in cross-hatching in the FIGURE. An elongated portion 14 is used essentially as a coilform as well as serving its usual surgical function. Another portion 16, when installed, lies within the femur.

A first coil 13 is wound around the elongated portion. A second coil 13 may also be wound around the elongated portion. These coils are connected with an integrated circuit chip 11. In an exemplary embodiment the two coils are decoupled (relative to each other) to at least some extent, for example by being orthogonal to each other or at least by being non-coplanar.

The practical effect is that an antenna and base station system may be employed to exchange messages with the chip. The functional distance may well be at least a foot or two. This permits tracking locations of such instruments.

The teachings of the invention also may help with tracking locations of metal tools, such as wrenches, which likewise are impossible to track using traditional RFID tags.

The frequencies employed are less than 450 kilohertz and preferably less than 150 kilohertz.

The implant instruments require heat sterilization at temperatures more than 100 C. Thus, the tags in these applications are, in an exemplary embodiment, be battery-less radiating tags (BLTs) such as those set forth in U.S. application Ser. No. 11/419,750, filed May 22, 2006, which application is incorporated herein by reference for all purposes.

The base station apparatus employed for communication with the implant assembly may, in an exemplary embodiment, be that set forth in U.S. application Ser. No. 11/462,981, filed Aug. 7, 2006, which application is incorporated herein by reference for all purposes.

An exemplary coil may be 12 mm in diameter, with 200 turns of copper wire. Alternatively the coil may be saddle shaped, about 25 mm in diameter.

### EXAMPLE

A 12 mm coil on an actual instrument was able to be read from two feet or more.

Those skilled in the art will have no difficulty devising myriad obvious improvements and variations upon the invention, all of which are intended to be encompassed within the claims which follow.

1. A metal surgical implant assembly comprising:
a metal surgical implant with at least one elongated portion;
a first antenna wrapped around the at least one elongated portion;
the first antenna electrically coupled with circuitry;
the first antenna and circuitry tuned to a frequency below 450 kilohertz;
the assembly substantially sterile.

2. The assembly of claim 1 wherein the surgical implant comprises a hip implant.

3. The assembly of claim 1 wherein the surgical implant is stainless steel.

4. The assembly of claim 1 wherein the surgical implant is titanium.

5. The assembly of claim 1 further comprising a second antenna also wrapped around the at least one elongated portion;
the second antenna also electrically coupled with the circuitry;
the second antenna and circuitry tuned to a frequency below 450 kilohertz.

6. The assembly of claim 5 wherein the frequency to which the second antenna and circuitry is tuned is different from the frequency to which the first antenna and circuitry is tuned.

7. A metal tool comprising:
a metal tool with at least one elongated portion;
a first antenna wrapped around the at least one elongated portion;
the first antenna electrically coupled with circuitry;
the first antenna and circuitry tuned to a frequency below 450 kilohertz.

8. The assembly of claim 7 wherein the tool comprises a wrench.

9. The assembly of claim 7 wherein the tool is steel.

10. The assembly of claim 7 further comprising a second antenna also wrapped around the at least one elongated portion;
the second antenna also electrically coupled with the circuitry;
the second antenna and circuitry tuned to a frequency below 450 kilohertz.

11. The assembly of claim 10 wherein the frequency to which the second antenna and circuitry is tuned is different from the frequency to which the first antenna and circuitry is tuned.
